(19) United States
(12) Patent Application Publication
(10) Pub. No.: US 2025/0262182 A1
(43) Pub. Date: Aug. 21, 2025

(54) Beverage Composition and Process

(71) Applicant: Kurt H. Ruppman, Allen, TX (US)

(72) Inventor: Kurt H. Ruppman, Allen, TX (US)

(21) Appl. No.: 19/197,592

(22) Filed: May 2, 2025

Related U.S. Application Data
(63) Continuation-in-part of application No. 17/398,827, filed on Aug. 10, 2021.
(60) Provisional application No. 63/064,391, filed on Aug. 11, 2020.

(51) Int. Cl. A61K 31/225; A61K 9/00; A61K 31/198; A61K 33/00; A61K 45/06; A61P 7/00; A61P 9/10; A61P 25/28

(52) U.S. Cl. CPC A61K 31/225; A61K 9/0095; A61K 31/198; A61K 33/00; A61K 45/06; A61P 7/00; A61P 9/10; A61P 25/28

(57) ABSTRACT

A beverage comprising a hydrogen-infused, degassed water, a nonessential amino acid, and a dicarboxylic acid, are disclosed. The beverage can further include a natural sweetener, a tricarboxylic acid, an α-amino acid, an electrolyte, a caffeine composition, and a Vitamin B composition. Also disclosed are methods for making and using such beverage. The beverage provides benefits to health.

### CROSS-REFERENCE TO RELATED APPLICATIONS

This application is a continuation-in-part of U.S. patent application Ser. No. 17/398,827, filed on Aug. 10, 2021, which claims priority to and the benefit of U.S. Provisional Patent Application Ser. No. 63/064,391, filed on Aug. 11, 2020, the disclosures of which are fully incorporated by reference.

### FIELD OF THE INVENTION

This invention relates to hydrogen-infused beverages intended to provide multiple health benefits. The invention further relates to methods for manufacturing such beverages.

### BACKGROUND OF THE INVENTION

Increasing nitric oxide (NO) in the blood to enhance blood flow is considered healthy because it supports the cardiovascular and circulatory system in a human body. NO is a naturally occurring signaling molecule produced by the endothelium, the thin layer of cells lining the blood vessels. When produced in sufficient quantities, nitric oxide causes the smooth muscle in vessel walls to relax, leading to vasodilation. This widening of the blood vessels allows blood to flow more easily, reducing resistance and improving circulation to tissues throughout the body.

Improved blood flow means more oxygen and nutrients are delivered to working muscles, organs, and the brain. It also enhances the removal of waste products like carbon dioxide and metabolic byproducts. From a cardiovascular standpoint, this reduces the strain on the heart, as it can pump blood more efficiently through less constricted vessels. For the brain, better perfusion supports cognitive clarity and protects against ischemic events by ensuring steady oxygen delivery. In muscle tissue, increased blood flow can enhance exercise performance and speed recovery by supplying glucose and amino acids more efficiently and clearing out lactic acid more rapidly.

Furthermore, boosting NO levels through healthy means—such as L-citrulline supplementation, nitrate-rich foods, or regular exercise—may contribute to the maintenance of endothelial health and reduce the risk of vascular diseases like hypertension, atherosclerosis, and stroke. In this way, increasing NO is not just about temporary performance gains or improving circulation in isolation; it's about supporting a broader system of cardiovascular integrity and metabolic efficiency. By facilitating smooth, responsive, and well-regulated blood flow, nitric oxide plays a central role in maintaining long-term vascular health.

Nitric oxide is a gas composed of one molecule of nitrogen bonded to one molecule of oxygen. It serves as a critical signaling molecule in the cardiovascular and circulatory system in a human body. This essential molecule signals blood vessels to relax and expand, creating greater efficiency in the body, greater nutrient delivery, and greater oxygenation to every system, organ and tissue in the body. The nitric oxide also helps in improving athletic performance.

Generally, diet and exercise play a large role in stimulating the natural production of nitric oxide. The nitric oxide stimulating foods or foods that are high in dietary nitrites/nitrates with regular exercise helps to promote nitric oxide production in the human body. Unfortunately, in today's world of refined foods these options may not be enough, especially for the athletes and those suffering from certain medical conditions.

Hydrogen water is said to increase energy, reduce inflammation, and reduce recovery times after workouts. Hydrogen water is regular water into which hydrogen has been infused. According to some resources, adding hydrogen gas to the water increases its anti-inflammatory and antioxidant properties when consumed. Hydrogen water has also been touted for its ability to increase energy, slow down the aging process, and improve muscle recovery after a workout.

Although nitric oxide can directly kill pathogens, it is also critical for immune function and can inhibit viral replication. However, nitric oxide in certain quantities or inhaled in an uncontrolled manner as a gas can also induce lethal cellular injury under stressed conditions. In contrast, molecular hydrogen helps regulate nitric oxide production and attenuates many of the harmful effects. In addition, hydrogen water typically referred to in the literature has limited beneficial effects in that they are short-lived, lasting less than a few hours.

Therefore, there is a need for supplements that promote the controlled production and/or maintenance of nitric oxide in the human body for an extended period of time.

### SUMMARY OF THE INVENTION

The present disclosure relates to a composition, a process for making the composition, and methods for use of the composition. In some embodiments, the composition comprises a beverage for enhancing athletic performance and acquiring other health benefits of nitric oxide for longer duration. In addition, the beverage of the present invention is useful in the treatment of medical conditions that benefit from increased blood flow for longer periods of time.

In one aspect, a method for increasing blood flow using a composition that increases nitric oxide content in blood is disclosed. The method comprises orally ingesting a composition comprising a nonessential amino acid (NAA), a dicarboxylic acid (DA), and molecular hydrogen (MH), at a first time and at a first blood flow rate. A second blood flow rate is achieved at a second time, wherein the second blood flow rate is greater than the first blood flow rate as measured by photoplethysmography at a fingertip. In some embodiments, the weight ratio of NAA to DA is in a range of from 0.8:1 to 375:1, and the ratio of the combined weight of NAA and DA to the weight of MH is in a range of from 250:1 to 21,000:1.

In another aspect, a composition useful for increasing NO content in blood and consequently increasing blood flow rate is disclosed. In some embodiments, the composition comprises a hydrogen-infused, degassed water having a molecular hydrogen content in a range of from 1 ppm to 6 ppm by weight. The composition further comprises a nonessential amino acid in an amount in a range of from 0.15 wt % to 3.0 wt %, based on the weight of the composition. The composition further comprises a dicarboxylic acid in an amount in a range of from 0.008 wt % to 0.180 wt %. Weight percentages are based on the total weight of the composition.

In yet another aspect, a process for making a beverage composition is disclosed.

In yet another aspect, a method for treatment of a patient having a medical condition is disclosed.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other compositions and/or processes for carrying out the purposes of the present invention. It should also be realized by those skilled in the art that such equivalent constructions do not depart from the spirit and scope of the invention as set forth in the appended claims. The novel features which are believed to be characteristic of the invention, both as to its composition and method of manufacture, together with further objects and advantages will be better understood from the following description.

### BRIEF DESCRIPTION OF THE DRAWINGS

The present disclosure is illustrated by way of example, and not by way of limitation, in the accompanying figure, in which:

FIG. 1 shows a graphical comparison of maximal aerobic power and functional threshold power before and after consuming the inventive beverage disclosed herein;

FIG. 2 shows a graphical comparison of maximal oxygen uptake before and after consuming beverage disclosed herein;

FIG. 3 shows a graphical comparison of average leg extension torque before and after consuming beverage disclosed herein;

FIG. 4 shows a graphical comparison of exhaled NO before and after consuming the inventive beverage disclosed herein;

FIG. 5 shows a graphical comparison of brain gauge before and after consuming the inventive beverage disclosed herein;

FIG. 6A and FIG. 6B show a graphical comparison of cognitive scores before and after consuming the inventive beverage disclosed herein;

FIG. 7 shows increased nitric oxide after consuming the inventive beverage disclosed herein;

FIG. 8 shows a comparison of the effects on blood flow over time induced by beverages containing infused hydrogen and malic acid and different amounts of citrulline as measured by photoplethysmography at a fingertip; and

FIG. 9 shows a comparison of the changes in total hemoglobin in frontal cortex induced by beverages containing infused hydrogen and malic acid and different amounts of citrulline as measured by functional near-infrared spectroscopy.

While the disclosed process and composition are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of”, “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used herein, “consisting essentially of” excludes additional material elements, but allows the inclusion of non-material elements that do not substantially change the nature of the invention.

As used herein, “consisting of” is closed and excludes all additional elements.

As used herein, “degassed potable fluid,” means a potable fluid from which volatile compounds and/or solids have been removed by any suitable process, including, but not limited to, reverse osmosis and vacuum distillation.

As used herein, “hydrogen,” means molecular hydrogen or hydrogen not chemically bonded to any atom other than hydrogen.

As used herein, “L-citrulline” is an amino acid, naturally occurring in food, such as watermelons and grapefruit, and also produced naturally in the human body as a byproduct of the enzymatic production of nitric oxide from the amino acid arginine, catalyzed by nitric oxide synthase, in particular in the kidneys. Citrulline is a non-essential amino acid, meaning that the body can manufacture it from other nutrients.

As used herein, “treated,” with respect to a fluid, means that the fluid is degassed to remove volatiles, filtered to remove solids, or both. For example, a treated water can refer to a degassed water, a filtered water, or water that has been both filtered and degassed.

As used herein, “wt %” means weight percent. Weight percentages are based on the total weight of the relevant composition unless otherwise specified.

### Athletic Performance and Nitric Oxide

Athletic performance is intimately tied to the body's ability to deliver oxygen and nutrients to working muscles, and nitric oxide (NO) plays a central role in that process. When NO production is inadequate, blood vessels remain more constricted, limiting blood flow to muscle tissues during exercise. This restriction can lead to reduced oxygen delivery and impaired removal of metabolic waste products like carbon dioxide and lactic acid. As a result, athletes may experience quicker onset of fatigue, diminished endurance, and slower recovery. Low NO levels are particularly limiting in high-intensity or endurance sports, where sustained oxygen delivery and efficient circulation are essential for optimal muscle performance and resilience under stress.

Conversely, producing and maintaining higher levels of NO in the bloodstream offers a variety of advantages for athletic performance. NO acts as a potent vasodilator, relaxing the smooth muscles that line blood vessels and allowing them to widen. This vasodilation improves circulation, which enhances the delivery of oxygen, glucose, and other nutrients to muscles while also accelerating the removal of waste products. Higher NO availability can therefore extend the time to fatigue, increase acrobic capacity, and support faster post-exercise recovery. In some studies, elevated NO levels have been linked to improved mitochondrial efficiency, further enhancing energy production at the cellular level. Moreover, athletes who are able to maintain higher NO through supplementation with compounds like L-citrulline, L-arginine, or dietary nitrates may experience improved “muscle pump,” endurance, and overall work capacity. In this way, nitric oxide serves as a critical biochemical ally in achieving peak physical performance.

It has been discovered that a beverage composition comprising certain relative and total amounts of a nonessential amino acid component and a dicarboxylic acid component and having a molecular hydrogen concentration within a certain range can produce a surprising increase in NO levels in the bloodstream and corresponding increases in blood flow rate. Furthermore, these increases can be maintained for an extended period of time. In some embodiments, the nonessential amino acid component comprises L-citrulline and/or L-arginine. In some embodiments, the nonessential amino acid component comprises L-citrulline. In some embodiments, the dicarboxylic acid component comprises malic acid.

Without wishing to be bound by any particular theory, it is believed that when molecular hydrogen is introduced into the body, such as by drinking hydrogen-infused water, it does not directly participate in the enzymatic reaction that converts L-arginine into nitric oxide (NO). That reaction, catalyzed by nitric oxide synthase (NOS), requires molecular oxygen and the coenzyme nicotinamide adenine dinucleotide phosphate (NADPH) to donate electrons. Hydrogen gas is chemically stable under physiological conditions and does not serve as an electron donor or reactant in that process. However, it is believed that molecular hydrogen may contribute indirectly to production and maintenance of NO in the bloodstream.

One of the key challenges to maintaining healthy NO signaling is the vulnerability of NO to rapid degradation by reactive oxygen species (ROS), such as but not limited to superoxide (O₂⁻). When NO encounters superoxide, they react to form peroxynitrite (ONOO⁻), a highly reactive and damaging molecule that not only reduces NO bioavailability but also contributes to oxidative stress and endothelial dysfunction. This reaction effectively nullifies the signaling benefits of nitric oxide. In other words, even if the citrulline-arginine pathway is generating NO efficiently, oxidative stress in the local tissue environment can negate its effects.

Although it doesn't donate electrons in the NOS reaction, it is believed that hydrogen gas can selectively scavenge the cytotoxic ROS without disturbing useful species like hydrogen peroxide (H₂O₂) that serve signaling roles. By reducing the presence of harmful radicals, molecular hydrogen can indirectly protect NO molecules that would otherwise be destroyed before they could exert their vasodilatory or anti-inflammatory effects.

Additionally, it is believed that hydrogen's antioxidative effects can help maintain the health of the endothelium, the inner lining of blood vessels where endothelial nitric oxide synthase (eNOS) resides. Oxidative damage to the endothelium impairs eNOS expression and activity, but when ROS are kept in check, the endothelial environment is more favorable to sustained NO production. This creates a supportive feedback loop: less oxidative stress improves NOS function, which leads to more NO, and it is further believed that hydrogen helps preserve that NO once it is produced.

In this way, it is believed that molecular hydrogen operates not as a primary reactant in nitric oxide synthesis but as a protective cofactor in the broader biochemical environment. Its presence may amplify the effectiveness of citrulline or arginine supplementation by preventing NO degradation and supporting vascular health, particularly under conditions of oxidative stress such as intense exercise, aging, or chronic disease.

The body produces nitric oxide (NO) through two distinct biochemical pathways: the arginine-nitric oxide synthase (NOS) pathway and the nitrate-nitrite-NO pathway. In the first, the amino acid L-arginine is converted into NO by the enzyme nitric oxide synthase, a process that requires oxygen and NADPH. This pathway is the primary source of NO under normal, oxygen-rich conditions and is active in endothelial cells, neurons, and immune cells. The second pathway begins with dietary nitrates—found in foods like beets and leafy greens—which are first converted to nitrite by oral bacteria and then to NO in the body, especially under low-oxygen or acidic conditions. This nitrate-nitrite-NO route serves as an important backup system when oxygen levels are low, such as during exercise or hypoxia. Both pathways ultimately support blood flow, oxygen delivery, and cellular signaling, but they are triggered and regulated differently based on the body's environment and physiological needs. Both pathways ultimately generate the same molecule—NO—and once that NO is in the system, it is subject to degradation by reactive oxygen species (ROS), especially superoxide (O₂⁻). That's where hydrogen gas comes in: it doesn't care how the NO was made. It simply acts as a selective antioxidant, neutralizing radicals that would otherwise destroy NO before it can signal properly.

In the arginine-NOS pathway, which requires oxygen and NADPH, NO is produced enzymatically, mainly in endothelial or neuronal tissue. But in oxidative environments (e.g., in metabolic syndrome, during intense exercise, or with aging), NO is often degraded quickly. Similarly, in the nitrate-nitrite-NO pathway, which occurs mostly under low oxygen or acidic conditions, NO is generated non-enzymatically. That NO is just as vulnerable to ROS.

Hydrogen, being extremely small and nonpolar, diffuses easily into cells and tissues and selectively neutralizes hydroxyl radicals (·OH) and peroxynitrite (ONOO⁻)—the latter being a direct result of NO reacting with superoxide. By reducing these radicals, H₂ preserves NO's half-life and bioavailability, helping it carry out its effects, whether that's vasodilation, anti-inflammation, or neurotransmission.

It is believed that hydrogen supports both pathways indirectly, by protecting the NO molecule itself, regardless of its origin. That's what makes combining citrulline (or arginine)+hydrogen or nitrates+hydrogen an attractive therapeutic or performance-enhancing strategy.

Without wishing to be bound by any particular theory, it is believed that when malic acid is added to citrulline, forming citrulline malate, it introduces a metabolic component that complements the nitric oxide pathway supported by citrulline. While malic acid does not participate directly in the production of nitric oxide, it is believed to play a significant role in cellular energy metabolism. Malate is a naturally occurring intermediate in the tricarboxylic acid cycle (TCA or Krebs cycle), which is the central energy-generating pathway in the mitochondria of cells. By feeding into this cycle, malate helps support the production of adenosine triphosphate (ATP), the molecule that fuels nearly all biological processes, including muscle contractions and recovery from exercise.

This boost in energy metabolism may help reduce muscle fatigue and improve endurance, particularly when paired with citrulline's ability to enhance blood flow through nitric oxide production. The vasodilatory effect of nitric oxide improves oxygen and nutrient delivery to working muscles, while malate supports the efficient use of those nutrients within the cell. Together, they create a synergistic effect: citrulline helps get oxygen and nutrients where they need to go, and malate helps cells extract and use that energy more effectively.

It is further believed that malate may play a role in buffering lactic acid accumulation during high-intensity exercise, potentially reducing post-exercise muscle soreness and improving recovery time. This function would complement citrulline's own role in the urea cycle, where it assists in the removal of ammonia—a byproduct of intense muscular activity that contributes to fatigue. While malic acid does not increase nitric oxide production on its own, its contribution to energy cycling and recovery processes makes it a valuable co-supplement.

### Treatment Method

In some embodiments, the present invention relates to a method for increasing blood flow rate for improved cardiovascular health, cognitive clarity, and/or improved athletic performance by increasing NO levels in the blood and/or maintaining increased NO levels in the blood. The method comprises orally ingesting a composition comprising a nonessential amino acid (NAA), a dicarboxylic acid (DA), and molecular hydrogen (MH), at a first time and at a first blood flow rate. A second blood flow rate is then achieved at a second time, wherein the second blood flow rate is greater than the first blood flow rate.

In some embodiments, the weight ratio of NAA to DA is greater than or equal to 0.8:1, greater than or equal to 2:1, or greater than or equal to 3:1. In some embodiments, the weight ratio of NAA to DA is less than or equal to 375:1, less than or equal to 200:1, or less than or equal to 16:1. In some embodiments, the weight ratio of NAA to DA is in a range of from 0.8:1 to 375:1, from 2:1 to 200:1, or from 3:1 to 16:1.

In some embodiments, the ratio of the combined weight of NAA and DA to the weight of MH is greater than or equal to 250:1, greater than or equal to 1,000:1, or greater than or equal to 2,000:1. In some embodiments, the ratio of the combined weight of NAA and DA to the weight of MH is in a range of less than or equal to 32,000:1, less than or equal to 18,000:1, or less than or equal to 4,000:1. In some embodiments, the ratio of the combined weight of NAA and DA to the weight of MH is in a range of from 250:1 to 32,000:1, from 1,000:1 to 18,000:1, or from 2,000:1 to 4,000:1.

In some embodiments, oral ingestion is completed in greater than or equal to 30 seconds, greater than or equal to a minute, or greater than or equal to 2 minutes. In some embodiments, oral ingestion is completed in less than or equal to 15 minutes, less than or equal to 10 minutes, or less than or equal to 5 minutes. In some embodiments, oral ingestion is completed in the range of from 30 seconds to 15 minutes, from 1 minute to 10 minutes, or from 2 minutes to 5 minutes.

In some embodiments, the ratio of the second blood flow rate to the first blood flow rate is greater than or equal to 1.1, greater than or equal to 1.2, greater than or equal to 1.3, or greater than or equal to 1.4. In some embodiments, the ratio of the second blood flow rate to the first blood flow rate is less than or equal to 2.0, less than or equal to 1.9, less than or equal to 1.8, or less than or equal to 1.7. In some embodiments, the ratio of the second blood flow rate to the first blood flow rate is in the range of from 1.1 to 2.0, in the range of from 1.2 to 1.9, in the range of from 1.3 to 1.8, or in the range of from 1.4 to 1.7.

In some embodiments, the difference between the second time and the first time is greater than or equal to 30 minutes, greater than or equal to 1 hour, greater than or equal to 1.5 hours, or greater than or equal to 2.0 hours. In some embodiments, the difference between the second time and the first time is less than or equal to 8.0 hours, less than or equal to 6.0 hours, less than or equal to 4.0 hours, or less than or equal to 3.0 hours. In some embodiments, the difference between the second time and the first time is in the range of from 30 minutes to 8.0 hours, in the range of from 1.0 hour to 6.0 hours, in the range of from 1.5 to 4.0 hours, or in the range of from 2.0 hours to 3.0 hours.

In some embodiments, the total amount of citrulline is greater than 1,000 mg, greater than or equal to 1,250 mg, or greater than or equal to 1,500 mg. In some embodiments, the total amount of citrulline is less than or equal to 10,000 mg, less than or equal to 7,500 mg, or less than or equal to 6,250 mg. In some embodiments, the total amount of citrulline is in the range of from 1,000 mg to 10,000 mg, in the range of from 1,250 mg to 7,500 mg, or in the range of from 1,500 mg to 6,250 mg.

### Beverage Composition

In some embodiments, the present invention relates to a beverage composition comprising the reaction product or product of, a mixture of a hydrogen-infused potable fluid, and a nonessential amino acid. In some embodiments, the beverage composition further comprises one or more of a natural sweetener, a dicarboxylic acid, a tricarboxylic acid, an α-amino acid, an electrolyte, a caffeine composition, and a Vitamin B composition.

### Hydrogen-Infused Potable Fluid

The beverages disclosed herein comprise a base potable fluid, which has been infused with hydrogen. In some embodiments, the potable fluid is degassed prior to infusion with hydrogen. In some embodiments, the potable fluid is water, tea, coffee, fruit juice, or water. In some embodiments, the potable fluid is degassed water.

Degassing can be accomplished by any suitable method, such as, but not limited to, reverse osmosis and vacuum distillation. Reverse osmosis is a process by which a solvent passes through a porous membrane in the direction opposite to that for natural osmosis when subjected to a hydrostatic pressure greater than the osmotic pressure. Vacuum distillation is distillation performed under reduced pressure, which allows the purification of compounds not readily distilled at ambient pressures. Regardless of the chosen method for degassing the base potable fluid, degassed potable fluids in some embodiments have a total dissolved solids (“TDS”), as determined by measuring the electrical conductivity of the potable fluid using a common TDS probe, of less than or equal to 150 ppm, 125 ppm, 100 ppm, 75 ppm, or 50 ppm.

In some embodiments, the base potable fluid is water. After degassing, the degassed potable fluid or degassed water is infused with hydrogen. Hydrogen water is made by bubbling pure hydrogen gas into water, by using electrolysis, by using a proton exchange membrane, by reaction with elemental magnesium, or by co-feed of hydrogen and a potable fluid, such as water, through a high-pressure nozzle, wherein the water is atomized into small droplets in the presence of highly concentrated hydrogen to increase hydrogen induction into the potable fluid.

Molecular hydrogen, in the context of hydrogen-infused water, refers to the diatomic hydrogen gas (H₂) that is physically dissolved into water without forming any new chemical compounds. Unlike the hydrogen already present in water molecules (H₂O), which is covalently bound to oxygen and chemically inert in that state, molecular hydrogen remains as free H₂ (i.e., two hydrogen atoms bonded together) and is suspended in solution much like carbon dioxide is in sparkling water. This form of hydrogen is biologically active in having antioxidant and/or anti-inflammatory properties. In hydrogen water, the goal is to enrich the liquid with dissolved H₂ at a sufficient concentration such that once ingested, molecular hydrogen can rapidly diffuse through tissues and selectively neutralize harmful reactive oxygen species (ROS), helping to reduce oxidative stress without interfering with beneficial signaling molecules.

In some embodiments, the potable fluid, such as degassed water, is infused with hydrogen in an amount greater than or equal to 0.5 ppm, greater than or equal to 0.8 ppm, greater than or equal to 1.0 ppm, greater than or equal to 1.5 ppm, greater than or equal to 1.6 ppm, greater than or equal to 1.7 ppm, greater than or equal to 1.8 ppm, greater than or equal to 1.9 ppm, greater than or equal to 2.0 ppm, greater than or equal to 2.1 ppm, greater than or equal to 2.2 ppm, greater than or equal to 2.3 ppm, greater than or equal to 2.4 ppm, greater than or equal to 2.5 ppm, by weight of the beverage composition. In some embodiments, the potable fluid, such as degassed water, is infused with hydrogen in an amount less than or equal to 8.0 ppm, less than or equal to 7.3 ppm, less than or equal to 6.7 ppm, less than or equal to 6.0 ppm, less than or equal to 5.4 ppm, less than or equal to 5.0 ppm, less than or equal to 4.7 ppm, less than or equal to 4.0 ppm, less than or equal to 3.0 ppm, or less than or equal to 2.0 ppm by weight of the beverage composition. Any upper endpoint can be combined with any lower endpoint to form a range. Any lower endpoint can be combined with another lower endpoint form a range. Any upper endpoint can be combined with another upper endpoint form a range. In some embodiments, the potable fluid, such as degassed water, is infused with hydrogen in an amount in the range of from 0.5 ppm, from 8.0 ppm, from 0.7 ppm to 6.9 ppm, from 0.8 ppm to 5.9 ppm, from 0.9 ppm to 4.8 ppm, from 1.0 ppm to 3.7 ppm, from 1.1 ppm to 2.7 ppm, or from 1.2 ppm to 1.6 ppm, by weight of the beverage composition.

### Nonessential Amino Acid

Nonessential amino acids are amino acids produced by the human body even if they are not ingested from food. Nonessential amino acids include but are not limited to: alanine, arginine, asparagine, aspartic acid, cysteine, glutamic acid, glutamine, glycine, proline, serine, and tyrosine including all of their enantiomers such as L-tyrosine. Nonessential amino acids support tissue growth and repair, immune function, red blood cell formation, and hormone synthesis. However, unlike essential amino acids, a healthy body can create these proteins if given enough protein sources with essential amino acids.

In some embodiments, the beverage composition comprises molecular hydrogen and a nonessential amino acid, wherein the nonessential amino acid(s) are L-citrulline and/or L-arginine. In some embodiments, the beverage composition comprises molecular hydrogen and a nonessential amino acid, wherein the nonessential amino acid is L-citrulline and/or L-arginine.

In some embodiments, the nonessential amino acid(s) are added to the mixture forming the beverage composition to produce a beverage comprising the nonessential amino acid(s) in an amount greater than or equal to 0.15 wt %, greater than or equal to 0.20 wt %, greater than or equal to 0.25 wt %, greater than or equal to 0.30 wt %, greater than or equal to 0.40 wt %, greater than or equal to 0.50 wt %, greater than or equal to 0.60 wt %, greater than or equal to 0.70 wt %, or greater than or equal to 1.0 wt %.

In some embodiments, the nonessential amino acid(s) are added to the mixture forming the beverage composition to produce a beverage comprising the nonessential amino acid(s) in an amount less than or equal to 8 wt %, less than or equal to 6 wt %, less than or equal to 4 wt %, less than or equal to 3 wt %, less than or equal to 2.75 wt %, or less than or equal to 2.5 wt %, based on the total weight of the mixture.

In some embodiments, the nonessential amino acid(s) are added to the mixture forming the beverage composition to produce a beverage comprising the nonessential amino acid(s) in an amount in a range of from 0.15 wt % to 8 wt %, from 0.15 wt % to 6 wt %, from 0.15 wt % to 4 wt %, from 0.15 wt % to 3 wt %, from 0.2 wt % to 2.75 wt %, from 0.25 wt % to 2.5 wt %, based on the total weight of the mixture.

Weight percentage in this context is based on the total weight of the beverage composition. In one embodiment, the nonessential amino acid added to the mixture is citrulline, and the beverage composition comprises citrulline in an amount in a range of from 0.15 wt % to 8 wt %, from 0.15 wt % to 6 wt %, from 0.15 wt % to 4 wt %, from 0.15 wt % to 3 wt %, from 0.2 wt % to 2.75 wt %, from 0.25 wt % to 2.5 wt %, based on the total weight of the mixture.

In some embodiments, a serving size of the beverage composition is about 250 ml. In some embodiments, the amount of nonessential amino acid added to such serving size in an amount greater than or equal to 375 mg, greater than or equal to 500 mg, greater than or equal to 625 mg, greater than or equal to 750 mg, greater than or equal to 1,000 mg, greater than or equal to 1,250 mg, greater than or equal to 1,500 mg, or greater than or equal to 1,750 mg.

In some embodiments, the amount of nonessential amino acid added to such serving size in an amount less than or equal to 20 g, less than or equal to 15 g, less than or equal to 10 g, less than or equal to 7.5 g, less than or equal to 6.2 g, or less than or equal to 5.0 g.

In some embodiments, the amount of nonessential amino acid added to such serving size in an amount in a range of from 625 mg to 20 g, from 750 mg to 15 g, from 1,000 mg to 10 g, from 1,250 mg to 7.5 g, from 1,500 mg to 6.2 g, or from 1,750 mg to 5.0 g.

It has been noted in the open literature that L-citrulline is generally well tolerated by most people at dosages up to 6 g. Some studies indicate that dosages in the range of 6 g to 10 g may cause mild gastrointestinal upset. In some instances, dosages in the range of 10 g to 15 g may cause stomach discomfort, bloating, or diarrhea. Dosages over 20 g may cause electrolyte imbalance and/or headaches due to blood vessel dilation.

### Natural Sweetener

Natural sweeteners include, but are not limited to, stevia, erythritol, monk fruit, acesulfame potassium, allulose, xylitol, and yacon. In some embodiments, the mixture comprising molecular hydrogen and a nonessential amino acid further comprises a natural sweetener selected from one or more of stevia, erythritol, monk fruit, acesulfame potassium, allulose, xylitol, and yacon. In some embodiments, the natural sweetener is added to the mixture in an amount greater than or equal to 0.01 wt %, greater than or equal to 0.05 wt %, or greater than or equal to 0.25 wt %, based on the total weight of the beverage composition. In some embodiments, the natural sweetener is added to the mixture in an amount less than or equal to 2.0 wt %, less than or equal to 1.0 wt %, or less than or equal to 0.5 wt %, based on the total weight of the beverage composition. In some embodiments, the natural sweetener is added to the mixture in an amount in the range of from 0.01 wt % to 2.0 wt %, from 0.05 wt % to 1.0 wt %, or from 0.25 wt % to 0.5 wt %, based on the total weight of the beverage composition.

Stevia is extracted from the leaves of a plant called Stevia rebaudiana. Several sweet compounds are found in stevia leaves, primarily stevioside and rebaudioside, both of which are hundreds of times sweeter than sugar, gram for gram. Stevia is very sweet but has virtually no calories. Stevia is believed to be beneficial in lowering high blood pressure and in lowering blood sugar levels in people with diabetes.

Erythritol is a sugar alcohol found naturally in certain fruits and a processed version is available in powdered form. It contains about 6% of the calories in an equal amount of sugar with 70% of the sweetness. Ingestion of erythritol does not spike blood sugar or insulin levels or affect levels of blood fats like cholesterol or triglycerides.

Monk fruit is a type of fruit native to Southeast Asia used to make a natural sweetener called monk fruit extract, which is free of calories and carbohydrates. Some research suggests that it may also help support better blood sugar management. Monk fruit also contains antioxidant compounds known as mogrosides, which studies have shown can reduce markers of inflammation.

Acesulfame potassium is about 200 times sweeter than common sugar.

Allulose is a low-calorie sweetener that has 70% of the sweetness of sugar. People with diabetes and obesity can benefit from this sugar substitute because it is low in calories and has little effect on blood sugar.

Xylitol is a sugar alcohol with a sweetness similar to that of sugar with about two-thirds the calories of sugar. Xylitol is reputed to have some benefits for dental health, to improve bone density, and to help support your gut.

Yacon syrup is harvested from the yacon plant, which grows natively in the Andes in South America. Yacon is very high in fructooligosaccharides, which function as soluble fibers that feed the good bacteria in the intestine.

It is understood that selection of specific or combination of various natural sweeteners can be combined to focus or design a beverage composition of the invention for different uses, effects or treatments.

### Dicarboxylic Acid

In some embodiments, the dicarboxylic acid is one or more of adipic acid, fumaric acid, and malic acid. In some embodiments, the dicarboxylic acid is added to the mixture in an amount greater than or equal to 0.008 wt %, greater than or equal to 0.012 wt %, greater than or equal to 0.016 wt %, greater than or equal to 0.020 wt %, greater than or equal to 0.026 wt %, or greater than or equal to 0.030 wt %, based on the total weight of the beverage composition. In some embodiments, the dicarboxylic acid is added to the mixture in an amount less than or equal to 0.18 wt %, less than or equal to 0.16 wt %, less than or equal to 0.14 wt %, less than or equal to 0.12 wt %, less than or equal to 0.10, or less than or equal to 0.08 wt %, based on the total weight of the beverage composition. In some embodiments, the dicarboxylic acid is added to the mixture in an amount in the range of from 0.008 wt % to 0.180 wt %, from 0.012 wt % to 0.160 wt %, from 0.016 wt % to 0.140 wt %, from 0.20 wt % to 0.120 wt %, from 0.20 wt % to 0.100 wt %, or from 0.030 wt % to 0.080 wt %, based on the total weight of the beverage composition. In some embodiments, the dicarboxylic acid is malic acid.

Malic acid is a natural supplement found in fruits like apples that promotes the creation of energy for human cells. Malic acid is an organic compound with the molecular formula C₄H₆O₅. It is a dicarboxylic acid that is made by all living organisms, contributes to the sour taste of fruits, and is used as a food additive. Malic acid has two stereoisomeric forms, though only the L-isomer exists naturally.

### Tricarboxylic Acid

In some embodiments, the tricarboxylic acid is one or more of citric acid, isocitric acid, aconitic acid, and propane-1,2,3-tricarboxylic acid. In some embodiments, the tricarboxylic acid is added to the mixture comprising molecular hydrogen and a nonessential amino acid, in an amount greater than or equal to 0.01 wt %, greater than or equal to 0.05 wt %, or greater than or equal to 0.25 wt %, based on the total weight of the beverage composition. In some embodiments, the tricarboxylic acid is added to the mixture comprising molecular hydrogen and a nonessential amino acid, in an amount less than or equal to 2.0 wt %, less than or equal to 1.0 wt %, or less than or equal to 0.5 wt %, based on the total weight of the beverage composition. In some embodiments, the tricarboxylic acid is added to the mixture comprising molecular hydrogen and a nonessential amino acid, in an amount in the range of from 0.01 wt % to 2.0 wt %, from 0.05 wt % to 1.0 wt %, or from 0.25 wt % to 0.5 wt %, based on the total weight of the beverage composition. In some embodiments, the tricarboxylic acid is citric acid.

Citric acid is a sharp-tasting crystalline acid present in the juice of lemons and other sour fruits. It is made commercially by the fermentation of sugar and used as a flavoring and setting agent. Citric acid is an organic compound with the chemical formula HOC (CH₂CO₂H)₂. In biochemistry, it is an intermediate in the citric acid cycle, which occurs in the metabolism of all aerobic organisms.

### α-Amino Acid

“α-amino acid,” as used herein, means amino acids which have the amine group attached to the carbon atom next to the carboxyl group, or the alpha position. These are also known as 2-, alpha-, or α-amino acids and have a generic formula:

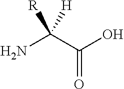

wherein R is an organic substituent (“side chain”).

These are generally L-stereoisomers (“left-handed” isomers), while a few D-amino acids (“right-handed”) occur in bacterial envelopes and in some antibiotics. In some embodiments, the α-amino acid has a hydrophilic nonacidic side chain on the α-carbon, such as, but not limited to serine, threonine, asparagine, and glutamine.

In some embodiments, the α-amino acid is one or more of serine, threonine, asparagine, and glutamine. In some embodiments, the α-amino acid is added to the mixture comprising molecular hydrogen and a nonessential amino acid, in an amount greater than or equal to 0.01 wt %, greater than or equal to 0.05 wt %, or greater than or equal to 0.25 wt %, based on the total weight of the beverage composition. In some embodiments, the α-amino acid is added to the mixture comprising molecular hydrogen and a nonessential amino acid, in an amount less than or equal to 2.0 wt %, less than or equal to 1.0 wt %, or less than or equal to 0.5 wt %, based on the total weight of the beverage composition. In some embodiments, the α-amino acid is added to the mixture comprising molecular hydrogen and a nonessential amino acid, in an amount in the range of from 0.01 wt % to 2.0 wt %, from 0.05 wt % to 1.0 wt %, or from 0.25 wt % to 0.5 wt %, based on the total weight of the beverage composition. In some embodiments, the α-amino acid is glutamine.

### Electrolyte

In some embodiments, the electrolyte is monopotassium phosphate. In some embodiments, the electrolyte is added to the mixture comprising molecular hydrogen and a nonessential amino acid, in an amount greater than or equal to 0.01 wt %, greater than or equal to 0.05 wt %, or greater than or equal to 0.25 wt %, based on the total weight of the beverage composition. In some embodiments, the electrolyte is added to the mixture comprising molecular hydrogen and a nonessential amino acid, in an amount less than or equal to 2.0 wt %, less than or equal to 1.0 wt %, or less than or equal to 0.5 wt %, based on the total weight of the beverage composition. In some embodiments, the electrolyte is added to the mixture comprising molecular hydrogen and a nonessential amino acid, in an amount in the range of from 0.01 wt % to 2.0 wt %, from 0.05 wt % to 1.0 wt %, or from 0.25 wt % to 0.5 wt %, based on the total weight of the beverage composition. In some embodiments, the α-amino acid is glutamine.

Monopotassium phosphate is the inorganic compound often used as a fertilizer, food additive, and buffering agent. The salt often cocrystallizes with the dipotassium salt as well as with phosphoric acid.

### Caffeine Composition

In some embodiments, the caffeine composition is natural green tea caffeine. In some embodiments, the caffeine composition is added to the mixture comprising molecular hydrogen and a nonessential amino acid, in an amount greater than or equal to 0.01 wt %, greater than or equal to 0.05 wt %, or greater than or equal to 0.25 wt %, based on the total weight of the beverage composition. In some embodiments, the caffeine composition is added to the mixture comprising molecular hydrogen and a nonessential amino acid, in an amount less than or equal to 4.0 wt %, less than or equal to 2.0 wt %, less than or equal to 1.0 wt %, or less than or equal to 0.5 wt %, based on the total weight of the beverage composition. In some embodiments, the caffeine composition is added to the mixture comprising molecular hydrogen and a nonessential amino acid, in an amount in the range of from 0.01 wt % to 2.0 wt %, from 0.05 wt % to 1.0 wt %, or from 0.25 wt % to 0.5 wt %, based on the total weight of the beverage composition. In some embodiments, the α-amino acid is glutamine.

Natural green tea caffeine also contains the amino acid L-theanine, which can work synergistically with caffeine to improve brain function 95 to 200 mg. According to a Journal of Food Science test of caffeine levels across commercial brands of green tea, the caffeine content in each 8 oz. cup varied by brand from 11 mg to 47 mg, and other sources record green teas that can contain upwards of 60 mg per brewed cup. Green tea may have a number of health benefits including, but not limited to, assisting in weight management, reducing skin inflammation, treating type 2 diabetes, and improving cardiovascular health. Green tea has one of the highest concentrations of antioxidants of any tea. It is naturally low in calories and contains less caffeine than black tea and coffee. In one embodiment, the beverage composition of the invention comprises a potable fluid, hydrogen, a nonessential amino acid, and green tea.

### Vitamin B Composition

In some embodiments, the Vitamin-B composition is selected from one or more of riboflavin (B2), niacin (B3), calcium pantothenate (B5), pyridoxine hydrochloride (B6), biotin (B7), folic acid (B9), and cyanocobalamin (B12). In some embodiments, the Vitamin-B composition is added to the mixture comprising molecular hydrogen and a nonessential amino acid, in an amount greater than or equal to 0.01 wt %, greater than or equal to 0.05 wt %, or greater than or equal to 0.25 wt %, based on the total weight of the beverage composition. In some embodiments, the Vitamin-B composition is added to the mixture comprising molecular hydrogen and a nonessential amino acid, in an amount less than or equal to 2.0 wt %, less than or equal to 1.0 wt %, or less than or equal to 0.5 wt %, based on the total weight of the beverage composition. In some embodiments, the Vitamin-B composition is added to the mixture comprising molecular hydrogen and a nonessential amino acid, in an amount in the range of from 0.01 wt % to 2.0 wt %, from 0.05 wt % to 1.0 wt %, or from 0.25 wt % to 0.5 wt %, based on the total weight of the beverage composition.

Vitamin B refers to several water soluble vitamins often found together in foods, all of which are necessary for normal growth and metabolism, but none of which are synthesized in adequate amounts by humans. The common forms of vitamin B include vitamin B1 (thiamine), B2 (riboflavin), B3 (niacin), B6 (pyridoxine) and B12 (cyanocobalamin).

Riboflavin (B2) works as an antioxidant, fighting damaging particles in the body known as free radicals. Antioxidants, such as riboflavin, can fight free radicals and may reduce or help prevent some of the damage they cause. Riboflavin is also needed to help the body change vitamin B6 and folate into forms it can use. It is also important for growth and red blood cell production.

Niacin (B3) is used to increase high-density lipoprotein (HDL) “good” cholesterol. Niacin and a related nutrient called niacinamide are used to treat or prevent niacin deficiency.

Calcium pantothenate (B5) is necessary for making blood cells, and it helps you convert the food you cat into energy.

Pyridoxine hydrochloride (B6) is essential to red blood cell, nervous system, and immune systems functions and helps maintain normal blood glucose levels.

Biotin (B7) is involved in a wide range of metabolic processes, both in humans and in other organisms, primarily related to the utilization of fats, carbohydrates, and amino acids.

Folic acid (B9) helps the body make healthy red blood cells and is found in certain foods.

Cyanocobalamin (B12) is important to maintain the health of your metabolism, blood cells, and nerves.

It is understood that any one of the above Vitamin-B can be used alone or in combination with the beverage composition comprising a potable fluid, molecular hydrogen and a nonessential amino acid.

### Other Beverage Embodiments

In some embodiments, the composition comprises the product or reaction product of a mixture of hydrogen-infused, degassed water, L-citrulline, erythritol, stevia, malic acid, citric acid, L-glutamine, monopotassium phosphate, riboflavin (B2), natural green tea caffeine, niacin (B3), calcium pantothenate (B5), pyridoxine hydrochloride (B6), biotin (B7), folic acid (B9), and cyanocobalamin (B12).

In some embodiments, the composition comprises the product or reaction product of a mixture of:
a treated water infused with hydrogen in an amount in the range of from 1.0 to 1.6 ppm by weight or 1.6 ppm to 5.4 ppm by weight;
L-citrulline in an amount in the range of from 0.15 wt % to 3.0 wt %;
erythritol, stevia; malic acid, citric acid, L-glutamine, monopotassium phosphate, and natural green tea caffeine, each in an amount greater than or equal to 0.01 wt %, greater than or equal to 0.05 wt %, or greater than or equal to 0.25 wt %; less than or equal to 2 wt %, less than or equal to 1 wt %, or less than or equal to 0.5 wt %; or in the range of from 0.01 wt % to 2 wt %, from 0.05 wt % to 1.0 wt %, or from 0.25 wt % to 0.5 wt %;
riboflavin (B2), in an amount greater than or equal to 650 ppm by weight, less than or equal to 1,300 ppm by weight, or in the range of from 650 ppm to 1,300 ppm by weight;
niacin (B3), in an amount greater than or equal to 8,000 ppm by weight, less than or equal to 16,000 ppm by weight, or in the range of from 8,000 ppm to 16,000 ppm by weight;
calcium pantothenate (B5), in an amount greater than or equal to 2,500 ppm by weight, less than or equal to 5,000 ppm by weight, or in the range of from 2,500 ppm to 5,000 ppm by weight;
pyridoxine hydrochloride (B6), in an amount greater than or equal to 650 ppm by weight, less than or equal to 1300 ppm by weight, or in the range of from 650 ppm to 1,300 ppm by weight;
biotin (B7), in an amount greater than or equal to 15 ppm by weight, less than or equal to 30 ppm by weight, or in the range of from 15 ppm to 30 ppm by weight;
folic acid (B9), in an amount greater than or equal to 200 ppm by weight, less than or equal to 400 ppm by weight, or in the range of from 200 ppm to 400 ppm by weight; and cyanocobalamin (B12), in an amount greater than or equal to 1.2 ppm by weight, less than or equal to 2.4 ppm by weight, or in the range of from 1.2 ppm to 2.4 ppm by weight;
wherein all weights are based on the weight of the mixture.

### Other Beverage Embodiments

In some embodiments, additional ingredients in the beverage composition include potassium, vitamin C, and cannabidiol.

### Process of Making Beverage Composition

In some embodiments, the process of making the beverage composition comprises infusing a potable fluid with hydrogen to produce a hydrogen-infused potable fluid and mixing the hydrogen-infused potable fluid with a nonessential amino acid, such as, but not limited to, L-citrulline, to produce an enhanced beverage composition of the invention.

In some embodiments, the process further includes degassing the potable fluid to remove volatiles and/or solids prior to infusing the potable fluid with hydrogen.

The process of either of the preceding process embodiments, further comprising mixing the enhanced beverage composition of the invention with one or more of a natural sweetener, a dicarboxylic acid, a tricarboxylic acid, an α-amino acid, an electrolyte, a caffeine composition, and a Vitamin-B composition.

### Beverage Advantages

Nitric oxide is a colorless, tasteless short-acting gas that helps blood vessels dilate, which in turn leads to an increased oxygen flow throughout the body. Nitric oxide, also called nitrogen oxide or nitrogen monoxide, is produced by almost every cell of the human body. Two amino acids, L-arginine and L-citrulline, boost nitric oxide production in the body. More specifically, the kidneys turn L-citrulline into L-arginine, which is a precursor to nitric oxide. Also, nitric oxide is produced in the human body from foods that contain nitrates, which are then converted to nitrites. Nitrites then are converted into nitric oxide in the body. Roughly 80 percent of dietary nitrates come from eating vegetables.

Nitric oxide is a gas that improves blood flow in areas of the lungs that are getting air, increasing the amount of oxygen in the blood stream. Nitric oxide plays key roles in maintaining normal vascular function and regulating inflammatory cascades that contribute to acute lung injury and acute respiratory distress syndrome. Nitric oxide also helps to support a healthy heart, boost the immune systems and increase exercise performance.

The main issue with nitric oxide is that it has a very short life and burns off very rapidly in the human body.

When adding at least 0.5 grams of a vegetable and/or fruit which contains nitrates or one of either of the amino acids, L-arginine or L-citrulline, and adding hydrogen with the aforementioned ingredients, the following new benefits can be achieved: increased blood flow that lasts for over 4 hours; limited to no increase in heart rate; limited to no increase in blood pressure; improved immune system; reduction of inflammation; increase sports performance; increased focus; and increased endurance.

Some disorders or physiological conditions can be medicated by promoting the production of nitric oxide in the human body. Increasing the production of nitric oxide can prevent, reverse, or limit the progression of disorders which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism and idiopathic or primary pulmonary hypertension or chronic hypoxia. However, nitric oxide can also induce lethal cellular injury under stressed conditions. Advantageously, molecular hydrogen helps regulate nitric oxide production and attenuates harmful effects. However, the inventive beverage composition is useful in treating the above disorders or physiological conditions. While the inventive beverage is not a cure, it is believed that the beneficial effects of extended nitric oxide release in the human body as a result of administering, drinking, the inventive beverage composition, can improve, reduce or minimize the listed disorders and physiological conditions. In a preferred embodiment it has been found that the beneficial effect of the beverage composition are enhanced when the beverage composition is consumed prior to ingesting solid food. It is best to drink the inventive beverage on an empty stomach.

### Certain Embodiments

In one aspect, disclosed herein is a method for increasing blood flow rate for improved cardiovascular health, cognitive clarity, and/or improved athletic performance by increasing NO levels in the blood and/or maintaining increased NO levels in the blood. In a first set of embodiments, the method comprises orally ingesting a composition comprising a nonessential amino acid (NAA), a dicarboxylic acid (DA), and molecular hydrogen (MH), at a first time and at a first blood flow rate. A second blood flow rate is then achieved at a second time, wherein the second blood flow rate is greater than the first blood flow rate. In further embodiments, the amount of the composition ingested is in a range of from 100 ml, 150 ml, or 200 ml to 300 ml, 350 ml, to 400 ml, 450 ml, 500 ml, 550 ml, or 600 ml. In some embodiments, for cans different in volume from the previously discussed 250 ml can, the amounts of each ingredient is adjusted in proportion to the ratio of a selected volume to the 250 ml benchmark volume.

A second set of embodiments comprises each embodiment of the first set of embodiments, wherein weight ratio of NAA to DA is:
a) greater than or equal to 0.8:1, greater than or equal to 2:1, or greater than or equal to 3:1;
b) less than or equal to 375:1, less than or equal to 200:1, or less than or equal to 16:1; or
c) in a range of from 0.8:1 to 375:1, from 2:1 to 200:1, or from 3:1 to 16:1.

A third set of embodiments comprises each embodiment of the first and second sets of embodiments, wherein the combined weight of NAA and DA to the weight of MH is:
a) greater than or equal to 250:1, greater than or equal to 1,000:1, or greater than or equal to 2,000:1;
b) less than or equal to 32,000:1, less than or equal to 18,000:1, or less than or equal to 4,000:1; or
c) in a range of from 250:1 to 32,000:1, from 1,000:1 to 18,000:1, or from 2,000:1 to 4,000:1.

A fourth set of embodiments comprises each embodiment of the first through the third sets of embodiments, wherein oral ingestion is completed in a time period:
a) greater than or equal to 30 seconds, greater than or equal to a minute, or greater than or equal to 2 minutes;
b) less than or equal to 15 minutes, less than or equal to 10 minutes, or less than or equal to 5 minutes; or
c) in the range of from 30 seconds to 15 minutes, from 1 minute to 10 minutes, or from 2 minutes to 5 minutes.

A fifth set of embodiments comprises each embodiment of the first through the fourth sets of embodiments, wherein the ratio of the second blood flow rate to the first blood flow rate is:
a) greater than or equal to 1.1, greater than or equal to 1.2, greater than or equal to 1.3, or greater than or equal to 1.4;
b) less than or equal to 2.0, less than or equal to 1.9, less than or equal to 1.8, or less than or equal to 1.7; or
c) in the range of from 1.1 to 2.0, in the range of from 1.2 to 1.9, in the range of from 1.3 to 1.8, or in the range of from 1.4 to 1.7.

A sixth set of embodiments comprises each embodiment of the first through the fifth sets of embodiments, wherein the difference between the second time and the first time is:
a) greater than or equal to 30 minutes, greater than or equal to 1 hour, greater than or equal to 1.5 hours, or greater than or equal to 2.0 hours;
b) less than or equal to 8.0 hours, less than or equal to 6.0 hours, less than or equal to 4.0 hours, or less than or equal to 3.0 hours; or
c) in the range of from 30 minutes to 8.0 hours, in the range of from 1.0 hour to 6.0 hours, in the range of from 1.5 to 4.0 hours, or in the range of from 2.0 hours to 3.0 hours.

A seventh set of embodiments comprises each embodiment of the first through the sixth sets of embodiments, wherein a serving size of the beverage composition is in a range of from about 100 ml, 150 ml, or 200 ml to about 300 ml, 350 ml, or 400 ml and:
a) the total amount of NAA or citrulline is:
   i) greater than or equal to 375 mg, greater than or equal to 500 mg, greater than or equal to 625 mg, greater than or equal to 750 mg, greater than or equal to 1,000 mg, greater than or equal to 1,250 mg, greater than or equal to 1,500 mg, or greater than or equal to 1,750 mg;
   ii) less than or equal to 20 g, less than or equal to 15 g, less than or equal to 10 g, less than or equal to 7.5 g, less than or equal to 6.2 g, or less than or equal to 5.0 g; or
   iii) in a range of from 625 mg to 20 g, from 750 mg to 15 g, from 1,000 mg to 10 g, from 1,250 mg to 7.5 g, from 1,500 mg to 6.2 g, or from 1,750 mg to 5.0 g;
b) the total amount of DA or malic acid is:
   i) greater than or equal to 20 mg, greater than or equal to 30 mg, greater than or equal to 40 mg, greater than or equal to 50 mg, greater than or equal to 65 mg, or greater than or equal to 75 mg;
   ii) less than or equal to 450 mg, less than or equal to 400 mg, less than or equal to 350 mg, less than or equal to 300 mg, less than or equal to 250 mg, or less than or equal to 200 mg; or
   iii) in a range of from 20 mg to 450 mg, from 30 mg to 400 mg, from 40 mg to 350 mg, from 50 mg to 300 mg, from 65 mg to 250 mg, or from 75 mg to 200 mg; or
c) a combination thereof.

In another aspect, disclosed herein is a composition comprising certain relative and total amounts of a nonessential amino acid component and a dicarboxylic acid component and having a molecular hydrogen concentration within a certain range. The composition can produce a surprising increase in NO levels in the bloodstream and corresponding increases in blood flow rate. In an eighth set of embodiments, the composition comprises a mixture of:
a) a hydrogen-infused, degassed water having a molecular hydrogen content:
   i) greater than or equal to 0.5 ppm, greater than or equal to 0.8 ppm, greater than or equal to 1.0 ppm, greater than or equal to 1.5 ppm, greater than or equal to 1.6 ppm, greater than or equal to 1.7 ppm, greater than or equal to 1.8 ppm, greater than or equal to 1.9 ppm, greater than or equal to 2.0 ppm, greater than or equal to 2.1 ppm, greater than or equal to 2.2 ppm, greater than or equal to 2.3 ppm, greater than or equal to 2.4 ppm, greater than or equal to 2.5 ppm, by weight of the beverage composition;
   ii) less than or equal to 8.0 ppm, less than or equal to 7.3 ppm, less than or equal to 6.7 ppm, less than or equal to 6.0 ppm, less than or equal to 5.4 ppm, less than or equal to 5.0 ppm, less than or equal to 4.7 ppm, less than or equal to 4.0 ppm, less than or equal to 3.0 ppm, or less than or equal to 2.0 ppm by weight of the beverage composition; or
   iii) in the range of from 0.5 ppm, from 8.0 ppm, from 0.7 ppm to 6.9 ppm, from 0.8 ppm to 5.9 ppm, from 0.9 ppm to 4.8 ppm, from 1.0 ppm to 3.7 ppm, from 1.1 ppm to 2.7 ppm, or from 1.2 ppm to 1.6 ppm, by weight of the beverage composition;
b) a nonessential amino acid in an amount:
   i) greater than or equal to 0.15 wt %, greater than or equal to 0.20 wt %, greater than or equal to 0.25 wt %, greater than or equal to 0.30 wt %, greater than or equal to 0.40 wt %, greater than or equal to 0.50 wt %, greater than or equal to 0.60 wt %, greater than or equal to 0.70 wt %, or greater than or equal to 1.0 wt % by weight of the beverage composition;
   ii) less than or equal to 8 wt %, less than or equal to 6 wt %, less than or equal to 4 wt %, less than or equal to 3 wt %, less than or equal to 2.75 wt %, or less than or equal to 2.5 wt %, by weight of the beverage composition; or
   iii) in a range of from 0.15 wt % to 8 wt %, from 0.15 wt % to 6 wt %, from 0.15 wt % to 4 wt %, from 0.15 wt % to 3 wt %, from 0.2 wt % to 2.75 wt %, from 0.25 wt % to 2.5 wt %, by weight of the beverage composition; and
c) a dicarboxylic acid in an amount:
   i) greater than or equal to 0.008 wt %, greater than or equal to 0.012 wt %, greater than or equal to 0.016 wt %, greater than or equal to 0.020 wt %, greater than or equal to 0.026 wt %, or greater than or equal to 0.030 wt %, based on the total weight of the beverage composition.
   ii) less than or equal to 0.18 wt %, less than or equal to 0.16 wt %, less than or equal to 0.14 wt %, less than or equal to 0.12 wt %, less than or equal to 0.10 wt %, or less than or equal to 0.08 wt %, based on the total weight of the beverage composition; or
   iii) in the range of from 0.008 wt % to 0.180 wt %, from 0.012 wt % to 0.160 wt %, from 0.016 wt % to 0.140 wt %, from 0.20 wt % to 0.120 wt %, from 0.20 wt % to 0.100 wt %, or from 0.030 wt % to 0.080 wt %, based on the total weight of the beverage composition.

A ninth set of embodiments comprises each embodiment of the eighth set of embodiments, wherein the nonessential amino acid is L-citrulline and/or L-arginine; wherein in further embodiments, the nonessential amino acid is L-citrulline.

A tenth set of embodiments comprises each embodiment of the eighth and ninth sets of embodiments, wherein the dicarboxylic acid is one or more members selected from the list consisting of: adipic acid, fumaric acid, and malic acid; wherein in further embodiments, the dicarboxylic acid is malic acid.

An eleventh set of embodiments comprises each embodiment of the eighth through the tenth sets of embodiments, wherein the mixture further comprises one or more members selected from the list consisting of: a natural sweetener, a tricarboxylic acid, an α-amino acid, an electrolyte, a caffeine composition, and a Vitamin-B composition.

A twelfth set of embodiments comprises each embodiment of the eighth through the tenth sets of embodiments, wherein the mixture further comprises two or more members selected from the list consisting of: a natural sweetener, a tricarboxylic acid, an α-amino acid, an electrolyte, a caffeine composition, and a Vitamin-B composition.

A thirteenth set of embodiments comprises each embodiment of the eighth through the tenth sets of embodiments, wherein the mixture further comprises three or more members selected from the list consisting of: a natural sweetener, a tricarboxylic acid, an α-amino acid, an electrolyte, a caffeine composition, and a Vitamin-B composition.

A fourteenth set of embodiments comprises each embodiment of the eighth through the tenth sets of embodiments, wherein the mixture further comprises four or more members selected from the list consisting of: a natural sweetener, a tricarboxylic acid, an α-amino acid, an electrolyte, a caffeine composition, and a Vitamin-B composition.

A fifteenth set of embodiments comprises each embodiment of the eighth through the tenth sets of embodiments, wherein the mixture further comprises five or more members selected from the list consisting of: a natural sweetener, a tricarboxylic acid, an α-amino acid, an electrolyte, a caffeine composition, and a Vitamin-B composition.

A sixteenth set of embodiments comprises each embodiment of the eighth through the tenth sets of embodiments, wherein the mixture further comprises a natural sweetener, a tricarboxylic acid, an α-amino acid, an electrolyte, a caffeine composition, and a Vitamin-B composition.

A seventeenth set of embodiments comprises each embodiment of the eleventh through the sixteenth sets of embodiments, wherein the natural sweetener is one or more members selected from the list consisting of: stevia, erythritol, monk fruit, acesulfame potassium, allulose, xylitol, and yacon.

An eighteenth set of embodiments comprises each embodiment of the eleventh through the seventeenth sets of embodiments, wherein the natural sweetener is present in an amount:
a) greater than or equal to 0.01 wt %, greater than or equal to 0.05 wt %, or greater than or equal to 0.25 wt %, based on the total weight of the beverage composition;
b) less than or equal to 2.0 wt %, less than or equal to 1.0 wt %, or less than or equal to 0.5 wt %, based on the total weight of the beverage composition; or
c) in the range of from 0.01 wt % to 2.0 wt %, from 0.05 wt % to 1.0 wt %, or from 0.25 wt % to 0.5 wt %, based on the total weight of the beverage composition.

A nineteenth set of embodiments comprises each embodiment of the eleventh through the eighteenth sets of embodiments, wherein the tricarboxylic acid comprises citric acid.

A twentieth set of embodiments comprises each embodiment of the eleventh through the nineteenth sets of embodiments, wherein the tricarboxylic acid is present in an amount:
a) greater than or equal to 0.01 wt %, greater than or equal to 0.05 wt %, or greater than or equal to 0.25 wt %, based on the total weight of the beverage composition;
b) less than or equal to 2.0 wt %, less than or equal to 1.0 wt %, or less than or equal to 0.5 wt %, based on the total weight of the beverage composition; or
c) in the range of from 0.01 wt % to 2.0 wt %, from 0.05 wt % to 1.0 wt %, or from 0.25 wt % to 0.5 wt %, based on the total weight of the beverage composition.

A twenty-first set of embodiments comprises each embodiment of the eleventh through the twentieth sets of embodiments, wherein the α-amino acid comprises L-glutamine.

A twenty-second set of embodiments comprises each embodiment of the eleventh through the twenty-first sets of embodiments, wherein the α-amino acid is present in an amount:
a) greater than or equal to 0.01 wt %, greater than or equal to 0.05 wt %, or greater than or equal to 0.25 wt %, based on the total weight of the beverage composition;
b) less than or equal to 2.0 wt %, less than or equal to 1.0 wt %, or less than or equal to 0.5 wt %, based on the total weight of the beverage composition; or
c) in the range of from 0.01 wt % to 2.0 wt %, from 0.05 wt % to 1.0 wt %, or from 0.25 wt % to 0.5 wt %, based on the total weight of the beverage composition.

A twenty-third set of embodiments comprises each embodiment of the eleventh through the twenty-second sets of embodiments, wherein the electrolyte comprises monopotassium phosphate.

A twenty-fourth set of embodiments comprises each embodiment of the eleventh through the twenty-third sets of embodiments, wherein the electrolyte is present in an amount:
a) greater than or equal to 0.01 wt %, greater than or equal to 0.05 wt %, or greater than or equal to 0.25 wt %, based on the total weight of the beverage composition;
b) less than or equal to 2.0 wt %, less than or equal to 1.0 wt %, or less than or equal to 0.5 wt %, based on the total weight of the beverage composition; or
c) in the range of from 0.01 wt % to 2.0 wt %, from 0.05 wt % to 1.0 wt %, or from 0.25 wt % to 0.5 wt %, based on the total weight of the beverage composition.

A twenty-fifth set of embodiments comprises each embodiment of the eleventh through the twenty-fourth sets of embodiments, wherein the caffeine composition comprises natural green tea.

A twenty-sixth set of embodiments comprises each embodiment of the eleventh through the twenty-fifth sets of embodiments, wherein the caffeine composition is present in an amount:
a) greater than or equal to 0.01 wt %, greater than or equal to 0.05 wt %, or greater than or equal to 0.25 wt %, based on the total weight of the beverage composition;
b) less than or equal to 2.0 wt %, less than or equal to 1.0 wt %, or less than or equal to 0.5 wt %, based on the total weight of the beverage composition; or
c) in the range of from 0.01 wt % to 2.0 wt %, from 0.05 wt % to 1.0 wt %, or from 0.25 wt % to 0.5 wt %, based on the total weight of the beverage composition.

A twenty-seventh set of embodiments comprises each embodiment of the eleventh through the twenty-sixth sets of embodiments, wherein the Vitamin-B composition is selected from one of more of the list consisting of: riboflavin (B2), niacin (B3), calcium pantothenate (B5), pyridoxine hydrochloride (B6), biotin (B7), folic acid (B9), and cyanocobalamin (B12).

A twenty-eighth set of embodiments comprises each embodiment of the eleventh through the twenty-seventh sets of embodiments, wherein Vitamin-B composition is present in an amount:
a) greater than or equal to 0.01 wt %, greater than or equal to 0.05 wt %, or greater than or equal to 0.25 wt %, based on the total weight of the beverage composition;
b) less than or equal to 2.0 wt %, less than or equal to 1.0 wt %, or less than or equal to 0.5 wt %, based on the total weight of the beverage composition; or
c) in the range of from 0.01 wt % to 2.0 wt %, from 0.05 wt % to 1.0 wt %, or from 0.25 wt % to 0.5 wt %, based on the total weight of the beverage composition.

A twenty-ninth set of embodiments comprises each embodiment of the eleventh through the twenty-eighth set of embodiments, wherein the nonessential amino acid is L-citrulline and/or L-arginine and the mixture further comprises one or more of the group consisting of: erythritol, stevia, malic acid, citric acid, L-glutamine, monopotassium phosphate, riboflavin (B2), natural green tea caffeine, niacin (B3), calcium pantothenate (B5), pyridoxine hydrochloride (B6), biotin (B7), folic acid (B9), and cyanocobalamin (B12).

A thirtieth set of embodiments comprises each embodiment of the eleventh through the twenty-ninth sets of embodiments, wherein the mixture further comprises cannabidiol.

In yet another aspect, disclosed herein is a beverage composition. In a thirty-first set of embodiments, the beverage composition comprises a mixture of:
a) a potable fluid comprising molecular hydrogen in an amount in the range of from 0.5 ppm to 6 ppm by weight;
b) L-citrulline in an amount in the range of from 0.15 wt % to 3.0 wt %;
c) erythritol, stevia; malic acid, citric acid, L-glutamine, monopotassium phosphate, and natural green tea caffeine, each in an amount less than or equal to 2 wt %;
d) riboflavin (B2), in an amount less than or equal to 1300 ppm by weight;
e) niacin (B3), in an amount less than or equal to 16,000 ppm by weight;
f) calcium pantothenate (B5), in an amount less than or equal to 5000 ppm by weight;
g) pyridoxine hydrochloride (B6), in an amount less than or equal to 1300 ppm by weight;
h) biotin (B7), in an amount less than or equal to 30 ppm by weight;
i) folic acid (B9), in an amount less than or equal to 400 ppm by weight; and
j) cyanocobalamin (B12), in an amount less than or equal to 2.4 ppm by weight;
wherein all weights are based on the weight of the mixture, and the potable fluid and/or the mixture is infused with molecular hydrogen.

A thirty-second set of embodiments comprises each embodiment of the thirty-first set of embodiments, wherein the potable fluid is water or treated water.

A thirty-third set of embodiments comprises each embodiment of the thirty-first and thirty-second sets of embodiments, wherein potable fluid and/or the mixture are infused with molecular hydrogen in an amount in the range of from 1.2 ppm to 3 ppm by weight.

In yet another aspect, disclosed herein is a process for making a beverage composition. In a thirty-fourth set of embodiments, the process for making a beverage composition comprises the steps of:
a) mixing a potable fluid with a nonessential amino acid to produce an enhanced beverage; and
b) infusing hydrogen into:
   i) the potable fluid prior to mixing, and/or
   ii) the enhanced fluid after mixing.

A thirty-fifth set of embodiments comprises each embodiment of the thirty-fourth set of embodiments, further comprising the step of treating the potable fluid before mixing.

A thirty-sixth set of embodiments comprises each embodiment of the thirty-fourth and thirty-fifth sets of embodiments, wherein the potable fluid is water, and the nonessential amino acid is L-citrulline and/or L-arginine.

A thirty-seventh set of embodiments comprises each embodiment of the thirty-fourth through thirty-sixth sets of embodiments, wherein level of hydrogen is added in an amount such that beverage composition contains hydrogen in an amount in the range of from 2 ppm and 6 ppm by weight.

A thirty-eighth set of embodiments comprises each embodiment of the thirty-fourth through thirty-seventh sets of embodiments, wherein the infusing comprises the use of a high-pressure nozzle.

In yet another aspect, disclosed herein is a method for treatment of a patient having a medical condition. In a thirty-ninth set of embodiments, the method for treatment of a patient having a medical condition comprises administering any composition of the eighth through the thirtieth sets of embodiments, wherein the medical condition is selected from one of the group consisting of: acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma, status asthmaticus or hypoxia, chronic pulmonary hypertension, bronchopulmonary dysplasia, chronic pulmonary thromboembolism, idiopathic or primary pulmonary hypertension and chronic hypoxia.

A fortieth set of embodiments comprises each embodiment of the thirty-ninth set of embodiments, wherein the administering takes place prior to the ingestion of any solid food.

The following examples illustrate the invention; however, those skilled in the art will recognize numerous variations within the spirit of the invention and scope of the claims. To facilitate a better understanding of the present invention, the following examples of preferred embodiments are given. In no way should the following examples be read to limit, or to define, the scope of the invention.

### Examples

The following examples are included to demonstrate preferred embodiments of the invention. It should be appreciated by those of skill in the art that the techniques disclosed in the examples which follow represent techniques discovered by the inventor to function well in the practice of the invention and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Physical Performance Testing

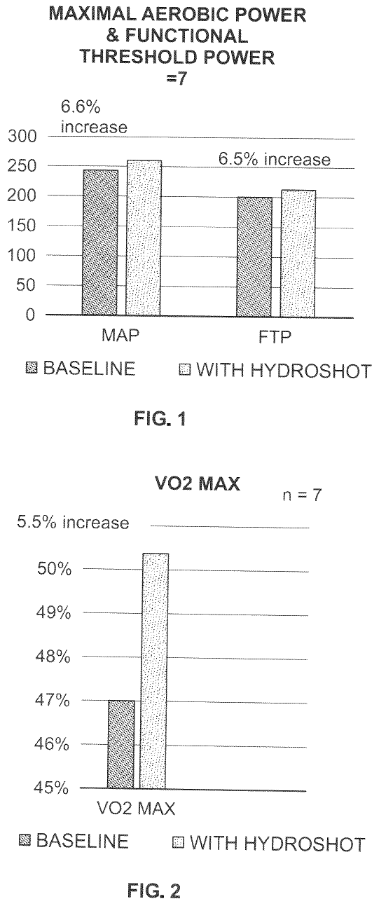

Testing was setup to determine possible improvements in maximal aerobic power (MAP), functional threshold power (FTP), and maximal oxygen uptake (VO2) using a cycle ergometer. Data is from drinking Hydro Shot vs baseline without Hydro Shot. Data generated at corpus performance sports facility in Dallas, Texas. Seven (7) subjects were included in the tests. Results are shown in FIG. 1 and FIG. 2.

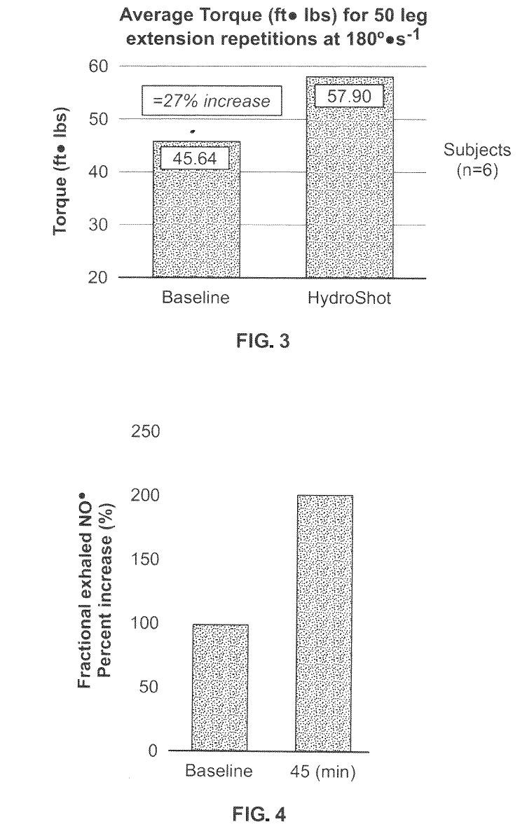

Testing was setup to determine possible improvements in leg extension repetition performance obtained from drinking Hydro Shot vs baseline without Hydro Shot. Data was generated at the Department of Kinesiology at Southern Utah University. Six (6) subjects were included in the tests. Results are shown in FIG. 3.

### Cognitive Testing

Testing was to determine possible sports performance improvements for speed, accuracy and focus drinking Hydro Shot vs baseline without drinking Hydro Shot. Data was generated using cortical metric's brain gauge tool at Corpus Performance Sports Facility in Dallas, Texas. Ten (10) subjects were included in the tests. Results are shown in FIG. 4.

The foregoing examples and related FIGS. 1-4 and results are published with additional details in: Effects of an H₂-infused, Nitric Oxide-Producing Functional Beverage on Exercise and Cognitive Performance, LeBaron, Kharman, and Mccullough, The Journal of Science and Medicine, Vol. 3, No. 2 (2021), and A novel functional beverage for COVID-19 and other conditions: Hypothesis and preliminary data, increased blood flow, and wound healing, LeBaron, Kharman, and Mccullough, Journal of Translational Science, Vol. 6, doi: 10.15761/JTS.1000380, ISSN: 2059-268X (2020)—the entire contents of both documents fully incorporated by reference herein.

### Traumatic Brain Injury Testing

Traumatic brain injuries may be caused by injuries from a number of sports, including soccer, boxing, football, baseball, lacrosse, skateboarding, hockey, and other high impact or extreme sports. These are particularly common in youth, explosive blasts, and other combat injuries.

An estimated 300,000 sports-related traumatic brain injuries (TBIs) of mild to moderate severity, most of which can be classified as concussions (i.e., conditions of temporarily altered mental status as a result of head trauma), occur in the United States each year. The proportion of these concussions that are repeat injuries is unknown. However, there is an increased risk for subsequent TBI among persons who have had at least one previous TBI.

Five-year outcomes of persons with TBI: 22% died, 30% became worse, 22% stayed same, and 26% improved. Data are US population estimates based on the TBIMS national database. Data refer to people 16 years of age and older who received inpatient rehabilitation services for a primary diagnosis of TBI.

Traumatic Brain Injury Diagnostic Methods include but not limited to:
MRI
Doppler Ultrasound
CTN Scan
UC Davis FIDWS
Impact
Cortical Metrics Brain Gauge

Beverages of the invention disclosed herein are useful for one or more therapeutic for TBI including:
Increases blood flow
Reduces inflammation
Improves focus
Improves memory
Boosts nitric oxide production for hours

Impact testing results before and after drinking hydrogen infused beverage are shown in TABLE 1:

| | Before | Before |
|---|---|---|
| | Hydrogen- | Hydrogen- |
| | infused | infused |
| | beverages | beverages |
| | disclosed | disclosed |
| | herein | herein |
| Test Method | (Hydro Shot) | (Hydro Shot) |
| Designed Memory | 60% | 77% |
| Learning Percent Correct | 63% | 83% |
| Percent of Letters Correct | 93% | 100% |
| Delayed Memory Percent | 58% | 71% |
| Correct | | |
| 3 Letters Counted Correctly | 6% | 11.2% |

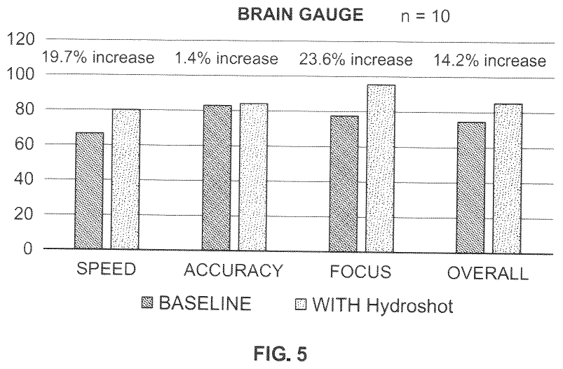

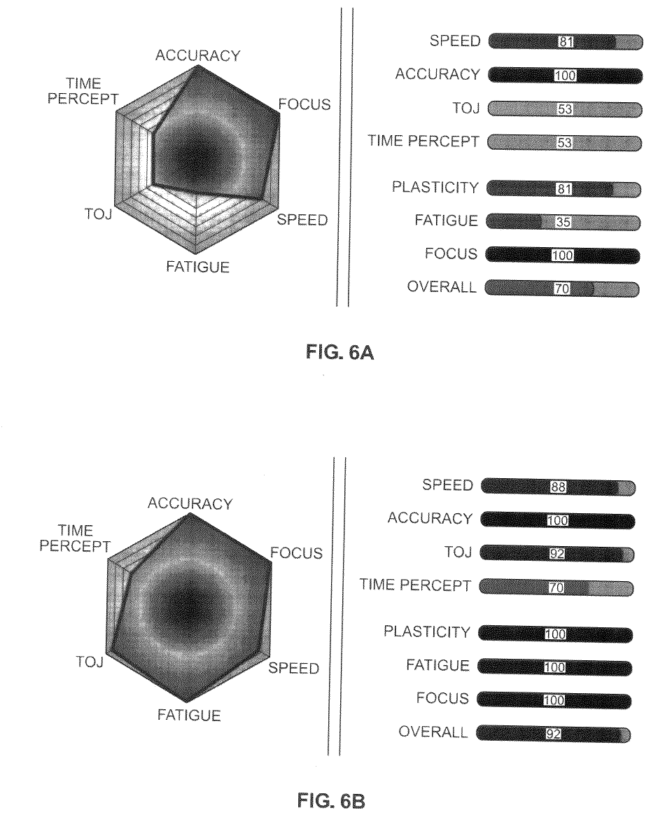

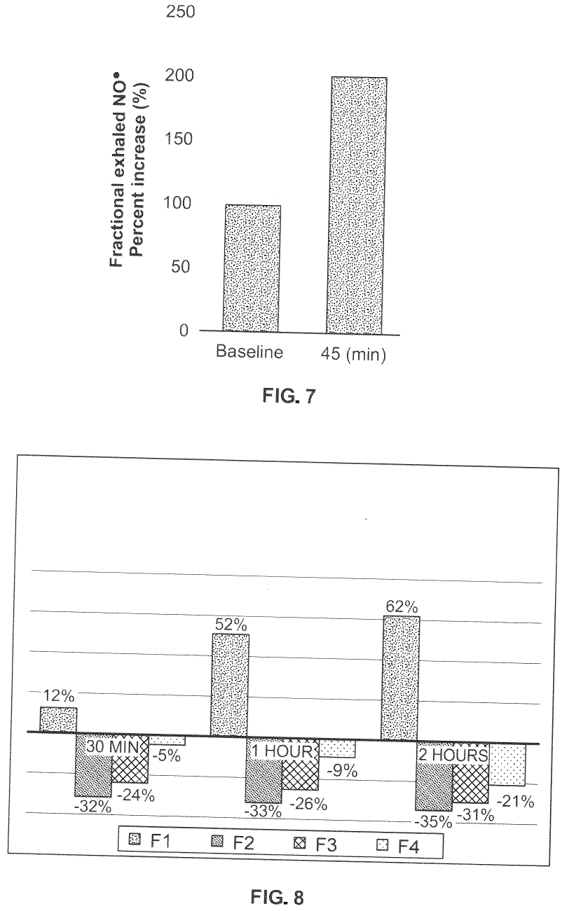

Cortical metrics brain gauge test results before and after drinking Hydro Shot are shown in FIGS. 5, 6A and 6B.

### Nitric Oxide Production Testing

Testing was performed to evaluate four hydrogen-enriched beverage formulations. The study compared a formulation of the beverage disclosed herein (F1) to three alternative formulations (F2-F4) as shown in TABLE 2 below. Formulations F1-F4 were all based on a 250 ml serving size. All formulations contained hydrogen at concentrations between 1.2-1.6 ppm by weight and malic acid in a range of from 50 mg to 300 mg (per 250 ml serving). All samples further contained caffeine and citric acid and additional ingredients in substantially the same amounts in order to isolate the effects of varying amounts of citrulline.

TABLE 2

| | Citrulline |
|---|---|
| Example | mg |
| 1 | 800 |
| 2 | 62.5 |
| 3 | 125 |
| 4 | 187.5 |

The testing utilized two complementary measurement technologies. Blood flow was measured using photoplethysmography. Cerebrovascular reactivity (CVR), reflecting the ability of blood vessels to dilate or constrict in response to a vasoactive stimulus, was measured using functional near-infrared spectroscopy (fNIRS).

### Blood Flow

Blood flow was measure using a proprietary Kyocera optical sensor that measures blood flow changes through fingertip readings. The system collected data at 40 Hz, capturing 40 samples per second, with each 5-second measurement period generating approximately 200 samples that were averaged to produce final readings. The device outputs data in CSV format in milliliters per minute (ml/min.) with timestamps for localized blood flow at the fingertip and heart rate measurements. When nitric oxide increases vasodilation, fingertip blood flow can double, triple, or more, depending on reactivity of the test subject's vessels. Increases in fingertip blood flow, as measured by photoplethysmography (PPG), are believed to be indicative of vasodilation and increased perfusion in the peripheral vasculature, which can reflect changes in the muscles of the test subject. Increased blood flow to the fingertips can indicate enhanced peripheral circulation as the body redirects blood to aid muscle recovery. Muscles may experience increased oxygen and nutrient delivery, facilitating repair and removal of metabolic byproducts like lactate. Testing was performed over a number of days by a single test subject with each formulation tested on separate days.

TABLE 3 shows raw data collected with the Kyocera device in milliliters per minute (ml/min.). The first column presents baseline measurements prior to ingestion of one of the formulations. Subsequent readings are shown for formulations F1-F4 at 30 minutes, 1 hour, and 2 hours. The raw data shows varying initial baseline values ranging from approximately 17.95 to 21.4. From this raw data, the first level of analysis involves calculating the difference between each time point measurement and the baseline value. The next analytical step converts these raw differences into percentage changes from baseline.

TABLE 3

| Formulation. | Baseline | 30 Min | 1 Hour | 2 Hours |
|---|---|---|---|---|
| F1 | 23.8 | 26.7 | 36.0 | 38.5 |
| F2 | 21.4 | 14.64 | 14.41 | 14.02 |
| F3 | 17.95 | 13.62 | 13.28 | 12.45 |
| F4 | 21.1 | 20.2 | 19.24 | 16.67 |

The processed data reveals distinct patterns among the formulations which are shown graphically in FIG. 8. Formulation F1 shows a unique positive trend, with increases of 12% at 30 minutes, progressing to 52% at 1 hour, and reaching 62% at 2 hours. In contrast, the other formulations demonstrate varying degrees of decrease from baseline. Formulation F2 shows consistent decreases ranging from −32% to −35%. Formulation F3 shows decreases from −24% to −31%. Formulation F4 shows a more gradual decline from −5% to −21%. FIG. 8 clearly illustrates the divergent patterns between inventive Formulation F1 and comparative Formulations F2-F4. The graph emphasizes the unique position of Formulation F1 as the only formulation showing positive percentage changes in blood flow, while the other formulations consistently show negative changes over the measured time periods.

### Cerebrovascular Reactivity

Cerebrovascular reactivity (CVR) was measured using functional near-infrared spectroscopy (fNIRS). Data was collected with a wearable device and a laboratory-grade CW6 system, available from TechEn Inc., Milford, MA. These systems collect data at 10-40 Hz depending on study parameters. The technology has been extensively validated through over 200 published research papers and is used by leading institutions including MIT, Harvard's MGH, Boston University, and Johns Hopkins APL. Test protocols similar to those used herein have been verified as shown in Chen, Donna Y., Xin Di, Keerthana Deepti Karunakaran, Hai Sun, Saikat Pal, and Bharat B. Biswal. “Delayed cerebrovascular reactivity in individuals with spinal cord injury in the right inferior parietal lobe: A breath-hold functional near-infrared spectroscopy study.” medRxiv (2024). https://doi.org/10.1101/2024.06.03.24307819. Testing was performed over a number of days by a single test subject with each formulation tested on separate days.

The CW6 data collection and analysis process involves measuring changes in oxyhemoglobin concentration during breath holding tests across multiple time periods. The data is collected using TechEn's functional near-infrared spectroscopy (fNIRS) technology, which measures changes in blood concentration at rates between 10-40 Hz depending on study parameters.

The wearable device was a headgear apparatus system designed for non-invasive monitoring of cerebral hemodynamics. It utilizes near-infrared light to measure changes in oxygenated and deoxygenated hemoglobin concentrations, providing insights into brain activity. Functional near-infrared spectroscopy (fNIRS) is an optical imaging technology that measures changes in blood concentration, specifically tracking variations in oxygenated and deoxygenated hemoglobin. The wireless wearable device and the CW6 system collected data at sampling rates ranging from 10 Hz to 40 Hz, enabling high-resolution temporal tracking of hemodynamic changes associated with neural activity. This frequency range supports both continuous monitoring and event-related designs in cognitive and physiological studies.

The testing protocol for the fNIRS using the CW6 system and the wireless wearable device measured cerebral hemodynamics-specifically, the changes in concentration of oxygenated hemoglobin (HbO₂) and deoxygenated hemoglobin (HbR) in the brain. These changes are proxies for neural activity, since regions of the brain that are more active require more oxygenated blood.

The breath holding test protocol using the CW6 system involves detailed monitoring of both oxygenated hemoglobin (HbO₂) and deoxygenated hemoglobin (HbR) concentrations in the blood. During the 15-second breath holding period, the levels of HbO₂ decrease as the body depletes its oxygen reserves, while HbR levels increase correspondingly. This creates a characteristic downward slope in the measurement curve for HbO₂.

When breathing resumes, there is a rapid physiological response where fresh oxygen intake leads to a quick increase in HbO₂ levels and a corresponding decrease in HbR concentration. This rapid change typically occurs within about 30 seconds after resuming breathing, though the exact timing can vary based on individual physiology and breathing patterns. The measurement shows a sharp upward slope for HbO₂ during this recovery phase.

The TechEn Wearable fNIRS device quantitatively measures these changes in the concentration of blood for oxy-hemoglobin, deoxy-hemoglobin and total hemoglobin during both brain activation tasks and rest periods. The testing protocol focuses measurements on the frontal cortex region, where the changes in HbO₂ and HbR concentrations can be precisely monitored. During normal breathing periods between breath holds, the measurements show more stable, horizontal lines for both HbO₂ and HbR levels, with the area under the curve (AUC) typically being greater than during breath-holding due to continuous oxygen intake.

The purpose of the testing was to compare the effects produced by ingestion of inventive Formulation F1 and comparative Formulations F2-F4. The testing measured quantitatively the changes in the concentration of blood for oxygenated hemoglobin, deoxygenated hemoglobin, and total hemoglobin, during brain activation tasks and at rest. Tests were performed on six (6) test subjects using a TechEn wearable functional near-infrared spectroscopy (fNIRS) device to measure these changes. The ability to safely measure and potentially increase the concentration of oxyhemoglobin may be significant for overall health, including sustained performance and focus.

The breath-holding test protocol is described below, wherein the increase in oxyhemoglobin was calculated after breath holding. When a person holds their breath for 15 seconds and then resumes normal breathing, the rate of change (increase) in oxyhemoglobin typically does change rapidly for a short period. This data was collected via the following protocol:
1) Test subject held breath for 15 seconds. During this time, the levels of oxyhemoglobin decreased as the body used up its oxygen reserves.
2) Test subject resumed normal breathing. Lungs rapidly took in fresh oxygen, which bound to hemoglobin in the blood, leading to a quick increase in oxyhemoglobin levels.
3) The process was repeated 6 times at each time interval that data was collected.

For about 30 seconds or even less, depending on individual physiology and breathing pattern, the increase in oxyhemoglobin was quite rapid as the test subject's body tried to replenish the oxygen that was used up during breath-holding. This rapid increase slowed down and stabilized as the body returned to normal oxygen level equilibrium. During breath-holding, the curve would slope downward, while normal breathing would show a more horizontal, stable line. The AUC during normal breathing is typically greater than during breath-holding due to continuous oxygen intake.

The analysis begins with raw data collection during breath holding tests, where measurements are taken at specific intervals including baseline, 30 minutes, 1 hour, 2 hours, and up to 4 hours after beverage consumption. During each test period, subjects undergo a 15-second breath holding cycle while the system monitors blood oxygenation changes. The data shows characteristic patterns where oxyhemoglobin levels initially decrease during breath holding, followed by rapid increases upon resuming normal breathing.

The breath holding test data analysis centers heavily on measuring and interpreting the area under the curve (AUC) during controlled breathing cycles. During each breath holding test, the data shows characteristic patterns where oxyhemoglobin levels initially decrease during the 15-second breath hold, followed by rapid increases upon resuming normal breathing. The AUC during normal breathing typically shows greater values than during breath-holding periods due to continuous oxygen intake.

The data interpretation process begins with analyzing the raw waveform data, which is collected at 10-40 Hz sampling rates. This high-frequency sampling ensures accurate capture of the rapid physiological changes during breath holding cycles. The waveform data shows distinct curve patterns—a downward slope during breath holding followed by a recovery curve upon breathing resumption. These curves are then integrated to calculate the total area under the curve (AUC), providing a quantitative measure of the hemodynamic response.

As shown in TABLE 4 below, when comparing the AUC calculations across formulations, Formulation F1 demonstrates consistently positive values ranging from 5,000 to 8,000 units throughout the four-hour testing period. Units are dimensionless and for comparison purposes only. Raw numbers are converted to oxygenated hemoglobin (HbO₂) and deoxygenated hemoglobin (HbR) concentrations in the blood by CW6 onboard algorithms and software. The interpretation of this data reveals significant differences in physiological response between formulations. While Formulation F1 maintains positive AUC values around 20,000 units, the other formulations show progressively negative responses. The comparative analysis becomes particularly striking when expressed as percentages as shown in TABLE 5 below. Formulation F2 shows an AUC approximately −8% relative to Formulation F1, while Formulations F3 and F4 demonstrate dramatically reduced AUC values at −273% and −306% respectively.

TABLE 4

| | | | | | | % Change |
|---|---|---|---|---|---|---|
| Formulation | 30 Minutes | One Hour | Two Hours | Four Hours | Totals | Relative to F1 |
| F1 | 7,250 | 6,875 | 8,065 | 7,025 | 29,215 | 0% |
| F2 | −550 | −1,100 | 700 | −1,300 | −2,250 | −8% |
| F3 | −20,325 | −1,9955 | −19,750 | −19,675 | −79,705 | −273% |
| F4 | −22,338 | −23,013 | −22,163 | −21,778 | −89,290 | −306% |

The detailed data processing includes baseline AUC calculations, as shown in the tables on page 9, where measurements are taken at specific time intervals (30 minutes, 1 hour, 2 hours, 4 hours). For example, the baseline AUC values range from 650 for the control measurement to −275 for certain formulations, demonstrating the significant variation in physiological responses between different beverages. This quantitative approach to AUC analysis provides a robust method for comparing the effectiveness of different formulations in maintaining blood oxygenation levels during and after breath holding challenges.

To monitor brain activity during a breath-holding task, the CW6 fNIRS device tracks changes in hemoglobin concentrations in the cerebral cortex, shedding light on how the brain responds to the physiological stress of breath-holding, specifically focusing on the increase in total oxyhemoglobin (HbO₂) in the frontal cortex, a key indicator of heightened blood flow and oxygenation triggered by rising carbon dioxide levels.

The CW6 system, using continuous-wave fNIRS, measures the relative changes in oxyhemoglobin concentration, typically reported in micromolar (μM) or micromoles per liter (μmol/L). These units reflect the concentration of HbO₂ in the cortical tissue being probed, calculated based on the differential absorption of near-infrared light (690-830 nm) by oxyhemoglobin versus deoxyhemoglobin. The modified Beer-Lambert law underpins this measurement, translating light attenuation into concentration changes. During breath-holding, hypercapnia induces vasodilation, increasing cerebral blood flow and HbO₂, which the CW6 captures as a rise in μM.

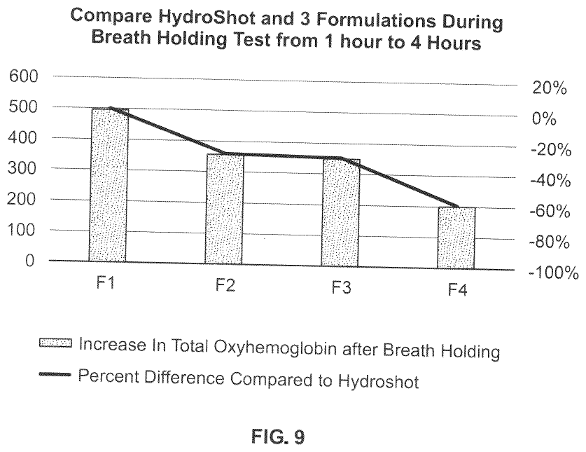

In the context of brain or muscle monitoring using fNIRS, total oxyhemoglobin refers to the concentration of hemoglobin in its oxygen-bound form—i.e., hemoglobin molecules (Hb) that are carrying oxygen (O₂). TABLE 5, below, shows the increase in total oxyhemoglobin (HbO₂) after the breath-holding tests. These results are shown graphically in FIG. 9.

TABLE 5

| | Increase | % |
|---|---|---|
| | In Total | Difference |
| | HbO₂ | Relative |
| Formulation. | (μM) | to F1 |
| F1 | 495 | 0 |
| F2 | 355 | −28 |
| F3 | 348 | −30 |
| F4 | 200 | −60 |

For the sake of brevity, only certain ranges are explicitly disclosed herein. However, in addition to recited ranges, any lower limit may be combined with any upper limit to recite a range not explicitly recited, as well as ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and/or testing procedures, to the extent they are not inconsistent with this text. As is apparent from the foregoing general description and the specific embodiments, while forms of the invention have been illustrated and described, various modifications can be made without departing from the spirit and scope of the invention.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the compositions, processes, and/or steps described in the specification. As one of the ordinary skill in the art will readily appreciate from the disclosure of the present invention, compositions, processes, and/or steps, presently existing or later to be developed that perform substantially the same function or achieve substantially the same result as the corresponding embodiments described herein, may be utilized according to the present invention. Accordingly, the appended claims are intended to include within their scope such compositions, processes, and/or steps.

What is claimed is:

1. A method for increasing blood flow rate, the method comprising:
orally ingesting a composition comprising a nonessential amino acid (NAA), a dicarboxylic acid (DA), and a hydrogen-infused, degassed water having a molecular hydrogen (MH), at a first time and at a first blood flow rate; and
achieving a second blood flow rate at a second time;
wherein:
the weight ratio of NAA to DA is in a range of from 0.8:1 to 375:1;
the ratio of the combined weight of NAA and DA to the weight of MH is in a range of from 250:1 to 32,000:1; and
the second blood flow rate is greater than the first blood flow rate.

2. The method of claim 1, wherein the amount of the composition ingested is in a range of from 100 ml to 600 ml.

3. The method of claim 1, wherein oral ingestion is completed in less than or equal to 15 minutes.

4. The method of claim 1, wherein the ratio of the second blood flow rate to the first blood flow rate is greater than or equal to 1.1.

5. The method of claim 1, wherein the difference between the second time and the first time is greater than or equal to 30 minutes.

6. The method of claim 1, wherein the total amount of citrulline is in a range of from 1,250 mg to 7,500 mg.

7. A composition comprising a mixture of:
a hydrogen-infused, degassed water having a molecular hydrogen content in a range of from 1 ppm to 6 ppm by weight; and
a nonessential amino acid in an amount in a range of from 0.15 wt % to 3 wt %; and
a dicarboxylic acid in an amount in a range of from 0.008 wt % to 0.180 wt %;
wherein weight percentages are based on the total weight of the composition.

8. The composition of claim 7 wherein the hydrogen-infused, degassed water has a molecular hydrogen content in an amount in the range of from 1.2 ppm to 3 ppm by weight.

9. The composition of claim 7, wherein the nonessential amino acid is L-citrulline and/or L-arginine.

10. The composition of claim 7, wherein the nonessential amino acid is L-citrulline.

11. The composition of claim 7, wherein the amount of nonessential amino acid is in the range of from 0.6 wt % to 2.5 wt %, based on the total weight of the composition.

12. The composition of claim 7, wherein the dicarboxylic acid is one or more members selected from the list consisting of: adipic acid, fumaric acid, and malic acid.

13. The composition of claim 7, wherein the dicarboxylic acid is malic acid.

14. The composition of claim 7, wherein the mixture further comprises:
a tricarboxylic acid in an amount in the range of from 0.01 wt % to 2.0 wt %;
a caffeine composition in an amount in the range of from 0.01 wt % to 2.0 wt %; or
a combination thereof.

15. The composition of claim 14, wherein the tricarboxylic acid is citric acid.

16. The composition of claim 7, wherein the mixture further comprises one or more members selected from the list consisting of: a natural sweetener, a tricarboxylic acid, an α-amino acid, an electrolyte, a caffeine composition, and a Vitamin-B composition.

17. The composition of claim 7, wherein the mixture further comprises two or more members selected from the list consisting of: a natural sweetener, a tricarboxylic acid, an α-amino acid, an electrolyte, a caffeine composition, and a Vitamin-B composition.

18. The composition of claim 7, wherein the mixture further comprises three or more members selected from the list consisting of: a natural sweetener, a tricarboxylic acid, an α-amino acid, an electrolyte, a caffeine composition, and a Vitamin-B composition.

19. The composition of claim 7, wherein the mixture further comprises a natural sweetener, a tricarboxylic acid, an α-amino acid, an electrolyte, a caffeine composition, and a Vitamin-B composition.

20. A method for treatment of a patient having a medical condition, the method comprising: administering a beverage composition comprising a mixture of a hydrogen-infused potable fluid and a nonessential amino acid, where the medical condition is selected from one of the group consisting of: acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma, status asthmaticus or hypoxia, chronic pulmonary hypertension, bronchopulmonary dysplasia, chronic pulmonary thromboembolism, idiopathic or primary pulmonary hypertension and chronic hypoxia.
